(19) United States
(12) Patent Application Publication
(10) Pub. No.: US 2007/0225733 A1
(43) Pub. Date: Sep. 27, 2007

(54) Device and Method

(75) Inventors: Andrew Barraclough, Berkshire (GB); Michel Wu, GuangDong Province (CN); Carl Dibnah, Hull (GB)

Correspondence Address: TRASK BRITT, P.O. BOX 2550, SALT LAKE CITY, UT, 84110, US

(73) Assignee: Reckitt Benckiser (UK) Limited, Slough Berkshire United Kingdom (GB)

(21) Appl. No.: 11/547,579

(22) PCT Filed: Apr. 6, 2005
(86) PCT No.: PCT/GB05/01338

§ 371 Date: Mar. 16, 2007

(30) Foreign Application Priority Data
Apr. 8, 2004 (GB) 0407988.5

(51) Int. Cl. A61B 17/50

(52) U.S. Cl. 606/133

(57) ABSTRACT

A device is provided to remove a depilatory composition and depilated hair 18 from the skin (20). The device (2) is moved in the direction of arrow (22) over the skin (20), and has fins (12) which remove the depilatory composition and depilated hair. During this movement non-depilatory compositions, for example a moisturiser and/or a fragrance, are applied to the skin from a impregnated sponge or fabric part (24) leading the fins and a impregnated sponge or fabric part (26) trailing the fins.

This invention relates to a device for removing a composition from the skin and to a method for such removal. The invention relates in particular, but not exclusively, to a device for use in a depilatory method.

Hair removing compositions are widely available. They may be epilatory and effect hair removal by mechanical action. They may be depilatory and effect hair removal by causing hair degradation or dissolution.

In the case of depilatory compositions the user applies these to their skin, leaves them for a predetermined interval to allow them to work and then removes them from the skin along with degraded or dissolved hair, usually using an article such as a scraper which is provided with the depilatory composition.

The depilatory compositions are effective but they may be perceived to have some undesirable skin effects, such as drying, as well as a residual odour. Also, when hair re-grows, this may lead to the formation of spots caused by trapped follicles.

Hence there is a need for a method of depilation which overcomes some of the problems which follow from the use of depilatory compositions on the skin.

According to a first aspect of the present invention, there is provided a device for removing a depilatory composition from the skin, the device comprising a head which, in use, is moved over the skin to effect removal of the depilatory composition and depilated hair and a handle to effect movement of the head, the head including at least one removal edge arranged, in use, to move over the skin to effect removal of the depilatory composition and depilated hairs, the head further including a source of a non-depilatory composition arranged to be applied to the skin when the head is moved over the skin.

In use the device is moved, such that the removal edge is traversed over the skin, to remove the depilatory composition and depilated hairs. In so doing it may break hairs weakened by exposure to the depilatory composition. However it would not normally break intact hairs, untreated by a depilatory composition. Preferably it has a scraping action, acting in the manner of a strigil. Whilst the device may effect the breaking of some hairs which have been weakened by the depilatory composition it cannot be used as a shaving device. Accordingly it does not have a cutting blade, and so it is a non-shaving device.

Preferably the head of the device is wider than the handle. Preferably the average width of the head is at least 50% greater than the average width of the handle, preferably at least 80% greater. For the purpose of this definition the average width of the handle is determined by taking width measurements at 1 cm intervals along the handle, starting 1 cm from its distal (free) end, summating the measurements and dividing by the number of measurements; and the average width of the head is determined by taking width measurements at 0.5 cm intervals along the head, starting 0.5 cm from its distal (free) end, summating the measurements and dividing by the number of measurements.

Preferably the maximum width of the head exceeds the maximum width of the handle. Preferably the maximum width of the head is at least 50% greater than the maximum width of the handle, preferably at least 80% greater. Preferably the maximum length of the handle exceeds the maximum length of the head, preferably by a factor of at least 3, more preferably by a factor of at least 5. The head will often be quite squat. Nevertheless it will be appreciated that what is meant by “length of the head” is its dimension in the same sense as the length of the handle.

Preferably then handle is slender. Preferably the head is not slender. Preferably it is broad.

Preferably the device has a stick-like or wand-like or rod-like handle, and a wider head or head part. Preferably the head is firmly carried by the handle in use; in the absence of an external force other than gravity or when merely touched there is preferably no tendency for it to be deflected. In use when bearing upon the skin it may articulate against a resistance force.

The non-depilatory composition may be a moisturiser.

The non-depilatory composition may be a fragrance.

The non-depilatory composition may include a water-soluble lubricant to prevent chafing, such as polyethylene oxide

The non-depilatory composition may be a composition which inhibits or slows the regrowth of hair.

The non-depilatory composition may be a buffer or acid which neutralises the alkaline pH of the depilatory composition.

The non-depilatory composition may be an oil. An oil may be both a moisturiser and a fragrance. An oil could be aromatherapy oil.

The non-depilatory composition may be any other material which could beneficially be applied to the skin, for example a colourant (such as a chemical “tanning” product), a soap, an exfoliating agent, an insect repellent, an anti-inflammatory agent, an emollient, a cooling agent, a buffer or a deodorising agent.

The non-depilatory composition could be a solid, including a gel. The solid may wear down, dissolve or melt as it deposits on the skin. The non-depilatory composition may be formed from a water-insoluble skeleton or matrix comprising water soluble or dispersible ingredients which leach out of the water-insoluble matrix in use. By water insoluble is meant having a solubility in water at 25° C. of 0.01% by weight of water or less. By water soluble is meant having a solubility in water at 25° C. of 0.1% by weight of water or more.

The non-depilatory composition may be applied upstream of the removal edge(s) or downstream of the removal edge(s) or, when there are two or more removal edge(s), from between them. Preferably, it is applied downstream of the removal edge(s).

The source of the non-depilatory composition may, for example, comprise a compressible or resilient part such as a fabric ply, felt pad or sponge, may be a film-forming mechanical device, for example a roller, or one of more small recesses, for example in the form of grooves or wells, into which the composition was deposited in manufacture, and from which it is drawn, when moved over the skin.

In a preferred form of the invention, the non-depilatory composition is supplied in the form of a solid block or strip. Suitable strips are disclosed in U.S. Pat. No. 4,170,821 for use with shaving razors. For instance, a water-soluble lubricant such as polyethylene oxide, mixed with a water-insoluble polymer such as polystyrene, may be used to form a solid strip of non-depilatory composition. EP0772511 discloses further co-extruded strips of polymeric material which are suitable for use as the non-depilatory composition of the invention.

A suitable lubricating strip to prevent chafing comprises 40% Polyethylene oxide (PEO) of molecular weight around 5 million, 20% of PEO of molecular weight around 200,000, and 40% polystyrene. Part of the high molecular weight polyethylene may be replaced by other ingredients such as plant extract, deodorant, fragrance, preservative and colourant.

The removal edge may be supplied by a fin, preferably of an elastomeric material. More than one fin could be employed.

The fin(s) may be flexible. In use, the fin(s) may project beyond the head to a greater extent than the source of a material to be applied to the skin when the fin(s) just contacts the skin. The source is arranged to be brought into contact with the skin when the fin(s) is caused to be flexed.

The or each fin may include a surface facing the skin, which surface has an angle, in use, of less than 90° to the skin with respect to the intended direction of movement of the device.

Preferably, the head has an underside (facing the skin in use). This underside is generally flat and preferably carries the removal edge(s)—preferably fin(s)—and the source of the non-depilatory composition. Preferably, the removal edge(s) project(s) transversely from the underside of the head. Preferably, the leading edge of the head does not function as a removal edge.

According to a second aspect of the present invention there is provided a method of effecting depilation comprising the steps of:
applying a depilatory composition to the skin;
allowing the depilatory composition to remain on the skin for a predetermined period of time, and
moving a device over the skin to remove the depilatory composition and depilated hair from the skin and to apply a non-depilatory composition to the skin.

The device and/or the non-depilatory composition may be as defined above in relation to the first aspect.

The invention will now be further described, by way of example, with reference to the accompanying drawings, in which:

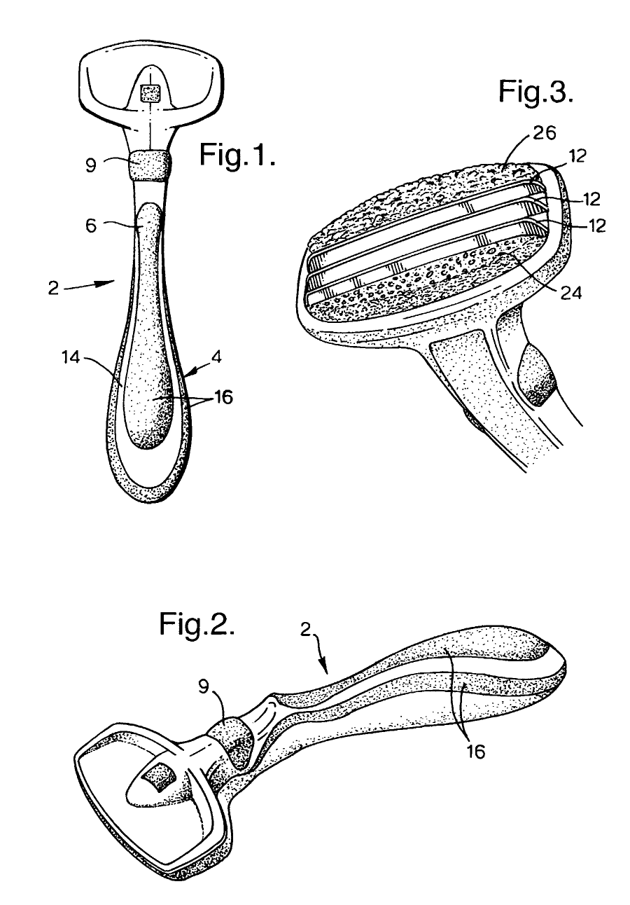

FIG. 1 is a plan view of a first embodiment of a device 2 of the invention, for removing a depilatory composition and depilated hair from a user's skin;

FIG. 2 is a front perspective view of the same device;

FIG. 3 is a view from underneath, of the head of the same device;

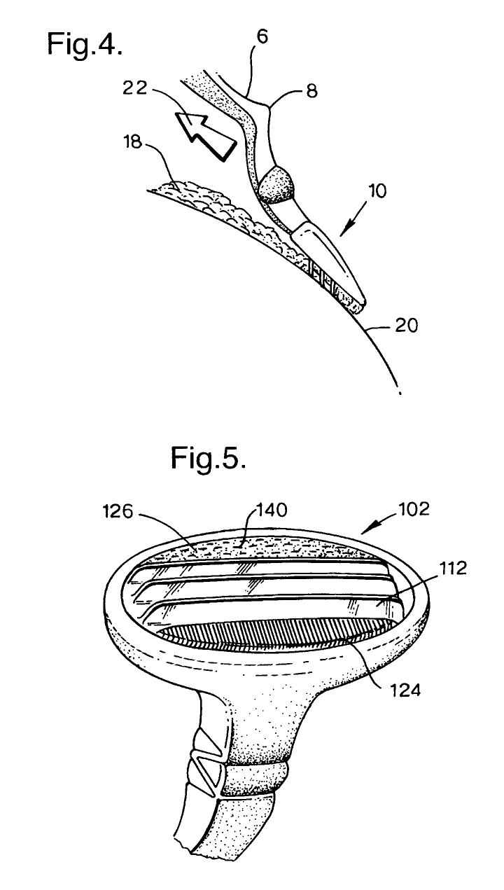

FIG. 4 is a detail showing the removal of depilatory composition and depilated hair the skin using the same device; and

FIG. 5 is a perspective view of the head of a second embodiment of device of the invention.

In the drawings shaded areas denote elastomeric material, soft to the touch, whereas areas shown denote plastics material, which is of higher modulus than the elastomeric material. The device 2 includes a handle 4 which is designed to be comfortably held by a user with a finger or thumb against a ramped portion 6, immediately adjoining a pronounced peak or step 8 in the handle. The handle is able to flex slightly about the step 8, and also about an elastomeric packing 9 between the step 8 and a head 10. The device is somewhat arched. In use, the arch faces the skin and, as the user presses down on it, it can flex slightly, leading to improved sensation of control.

As shown in FIG. 3, the head 10 includes three parallel, closely spaced fins. Each fin is straight and extends across substantially the full width of the head, and each is approximately 35 mm in length. The fins 12 are of elastomeric material. The fins 12 may be integrally moulded with the handle (which comprises a plastics section 14 and elastomeric sections 16 or may be added separately). The plastics material 14 extends down past the step 8 and forms the top of the head 10. The elastomeric material extends down beneath the step 8 towards the head 10.

As seen in FIG. 4, depilatory composition (typically containing potassium thioglycolate as active ingredient) is applied to the skin and left for a predetermined time. After that time, a user, holding the handle 4, pulls the head across the surface of the skin in the direction of arrow 22 (FIG. 4). This removes depilatory composition and depilated hair with the fins 12 also acting to detach weakened hairs from the skin, by a mild to moderate scraping action. It can be seen from FIGS. 3 and 4 that the fins project perpendicularly from the generally flat underside of the head but that they flex, during use, such that their surfaces which face the skin 20 are less than 90° with respect to the direction of movement 22.

The underside of the head 10 is also provided with a leading section 24 and a trailing section 26. The terms “leading” and “trailing” are used to denote the position of these sections relative to the fins, when the head is moved, in use, as shown by arrow 22. These sections project slightly from the underside of the head but not to the extent that the fins project, when in the relaxed position shown in FIG. 3. However, it can be seen from FIG. 4 that, when the fins flex, these sections 24, 26 may come into contact with the skin.

Either or both of the sections 24 and 26 may be comprised by a slightly compressible material such as sponge and impregnated with or otherwise able to deposit a non-depilatory composition onto the skin, in the same movement in which depilatory composition and depilated hair are removed.

There may be more than the number of sections that are shown either in advance of the fins with respect to the direction of the movement or trailing the blades or both. Alternatively or additionally, such sections may be provided between the fins.

The non-depilatory compositions may, for example, be selected from any one or more of the following substances: oils, moisturisers, perfumes, colourants, soaps, exfoliants, lubricants, anti-inflammatories, hair growth inhibitors, and insect repellents. In this embodiment, however, the leading section 24 is of sponge impregnated with a mild liquid soap to lubricate the movement of the fins and the trailing section 26 is of non-woven material impregnated with a dual action moisturiser/fragrance.

The device shown in FIG. 5 is for the same purpose. The head 102 is similar, but differs in a few respects. It is oval in shape rather than spade-like. However, it is still generally flat, and its underside also carries three elastomeric fins 112, a leading section 124 and a trailing section 126. In this embodiment, the leading section 124 is of a needlecord fabric material saturated with a skin lubricating/conditioning oil and the trailing section 126 is of rubber formed with pimples 140, to provide a massaging sensation. In this embodiment, the trailing section does not release a composition onto the skin.

1. A non-shaving device for removing depilatory composition from the skin, the device comprising
a head which has an underside (facing the skin in use) including at least one removal edge which projects transversely from the underside of the head, the removal edge being arranged such that, in use, it is moved over the skin to effect removal of the depilatory composition and depilated hair, and
a handle to effect movement of the head,
the head further including a source of a non-depilatory composition which is carried on the underside and is arranged to be applied to the skin when the head is moved over the skin.

2. A device according to claim 1 wherein the non-depilatory composition is a solid.

3. A device as claimed in claim 1 in which the non-depilatory composition is selected from at least one of the following: moisturiser, fragrance, soap, colourant, insect repellent, exfoliating agent, lubricant, anti-inflammatory, hair-regrowth inhibitor, and deodorising agent.

4. A device as claimed in claim 1 in which a said source is provided upstream of the removal edge.

5. A device as claimed in claim 1 in which a said source is provided downstream of the removal edge.

6. A device as claimed in claim 1 in which the removal edge is a flexible fin, which in its relaxed condition projects from the head to a greater extent than the source of the non-depilatory composition, but which in use flexes to allow the source to contact the skin.

7. A device as claimed in claim 1 in which the underside is generally flat.

8. A device as claimed in claim 1, having a plurality of removal edges.

9. A method of effecting depilation comprising the steps of:
applying a depilatory composition to the skin;
allowing the depilatory composition to remain on the skin for a predetermined period of time, and
moving a device over the skin to remove the depilatory composition and depilated hair from the skin and to apply a non-depilatory composition to the skin.

10. A device as claimed in claim 2 in which the non-depilatory composition is selected from at least one of the following: moisturiser, fragrance, soap, colourant, insect repellent, exfoliating agent, lubricant, anti-inflammatory, hair-regrowth inhibitor, and deodorising agent.

11. A device as claimed in claim 2 in which a said source is provided upstream of the removal edge.

12. A device as claimed in claim 3 in which a said source is provided upstream of the removal edge.

13. A device as claimed in claim 2 in which a said source is provided downstream of the removal edge.

14. A device as claimed in claim 3 in which a said source is provided downstream of the removal edge.

15. A device as claimed in claim 4 in which a said source is provided downstream of the removal edge.

16. A device as claimed in claim 2 in which the removal edge is a flexible fin, which in its relaxed condition projects from the head to a greater extent than the source of the non-depilatory composition, but which in use flexes to allow the source to contact the skin.

17. A device as claimed in claim 3 in which the removal edge is a flexible fin, which in its relaxed condition projects from the head to a greater extent than the source of the non-depilatory composition, but which in use flexes to allow the source to contact the skin.

18. A device as claimed in claim 4 in which the removal edge is a flexible fin, which in its relaxed condition projects from the head to a greater extent than the source of the non-depilatory composition, but which in use flexes to allow the source to contact the skin.

19. A device as claimed in claim 5 in which the removal edge is a flexible fin, which in its relaxed condition projects from the head to a greater extent than the source of the non-depilatory composition, but which in use flexes to allow the source to contact the skin.

20. A device as claimed in claim 2 in which the underside is generally flat.
